(19) United States
(12) Patent Application Publication
(10) Pub. No.: US 2022/0220497 A1
(43) Pub. Date: Jul. 14, 2022

(54) TPR1 GENE RELATED TO LOW-TEMPERATURE TOLERANCE OF POMACEA, CODING PROTEIN AND APPLICATION OF SAME

(71) Applicant: CHINA JILIANG UNIVERSITY, Hangzhou (CN)

(72) Inventors: Guangfu LIU, Hangzhou (CN); Xiaoping YU, Hangzhou (CN); Pengjun ZHANG, Hangzhou (CN); Xuping SHENTU, Hangzhou (CN); Yipeng XU, Hangzhou (CN); Qianqian YANG, Hangzhou (CN); Peiying HAO, Hangzhou (CN)

(73) Assignee: CHINA JILIANG UNIVERSITY, Hangzhou (CN)

(21) Appl. No.: 17/709,453

(22) Filed: Mar. 31, 2022

(30) Foreign Application Priority Data
Jan. 14, 2022 (CN) 202210043810.X

(51) Int. Cl. C12N 15/82

(52) U.S. Cl. CPC C12N 15/8273

(57) ABSTRACT

A TPR1 gene related to a low-temperature tolerance of Pomacea, coding protein and application of the gene is disclosed. The disclosure belongs to the field of biotechnology. The present disclosure is rapid, effective and reproducible, and is an important complement to the TPR1 gene family. Pomacea is an important invasive organism, the disclosure is helpful for developing novel biological pesticide, and the low-temperature tolerance of the organism is reduced by blocking the expression of TPR1 gene in Pomacea, so as to control the further northward invasion of Pomacea. Meanwhile, by interfering with the expression of TPR1 gene, the hatching rate of Pomacea eggs can be significantly reduced, and the hatching period of eggs can be prolonged, thereby reducing the harm of Pomacea invasion.

### CROSS-REFERENCE TO RELATED APPLICATIONS

This patent application claims the benefit and priority of Chinese Patent Application No. 202210043810.X filed on Jan. 14, 2022, the disclosure of which is incorporated by reference herein in its entirety as part of the present application.

### TECHNICAL FIELD

The present disclosure relates to the biotechnology field of freshwater mollusks, in particular to a TPR1 gene related to a low-temperature tolerance of Pomacea, coding protein and application of the gene.

### BACKGROUND ART

Pomacea, also known as large bottle snail and apple snail, is a vicious pest in the world. Originally from the Amazon River basin in South America, the snail was introduced to Asia in the early 1980s and was abandoned due to poor management and poor taste: Pomacea rapidly spread into the field, and some areas were flooded, causing serious damage to aquatic crops such as rice and water bamboo. Pomacea has been a serious agricultural pest in most provinces south of the Yangtze River due to its strong fecundity and fast spreading. Today. Pomacea is widely distributed in most provinces south of 30° N in China, including Zhejiang, Fujian, Guangdong, Hainan, Guangxi, Yunnan, Guizhou, Hunan, Jiangxi, Chongqing, Sichuan and Anhui provinces, the population density is very huge, which has a serious impact on agricultural production.

The reason why Pomacea is widely distributed in the middle and low latitudes of China is due to its tolerance to temperature, which is also the important reason why Pomacea can be successfully colonized in new habitats. Wada (2011) found that the probability of survival of Pomacea without cold acclimation for 2 days was less than 50% at 0° C., and all of them died at 5 days at 0° C.; after cold acclimation, more than 65% of Pomacea can survive for more than 5 days at 0° C. The cold tolerance of Pomacea has a seasonal regulation in summer, Pomacea can not survive for 5 days at 0° C. but in winter, the survival rate is nearly 100% under the same treatment, which indicates that Pomacea has a low temperature adaptation mechanism (Wada, 2007). Zhao Benliang et al. (2012) confirmed for the first time that the supercooling point of Pomacea invading South China exists, with an average temperature of around −7° C. This supercooling defense mechanism of Pomacea is beneficial to its adaptation to low temperature environment, and there is an ecological risk of further northward diffusion (Matsukura et al, 2009).

At present, there are many ways of prevention and control of Pomacea, such as artificial snails picking, artificial egg removing, duck and turtle co-breeding, which are restricted by the factors such as stability of effect, choice of implementation time and cost, etc, this kind of prevention and cure should not be used widely. Chemical control, using various chemicals to kill Pomacea. At present, the control of Pomacea is mainly based on chemical pesticides. Chemical control will pollute the environment to a certain extent, and have high toxicity to other aquatic organisms, moreover. Pomacea in some areas have resistance to chemical pesticides, which makes the control more difficult and costly. Some chemical pesticides can accumulate in human body after circulation, and produce accumulative toxicity, which brings great harm to human health. Therefore, it is very important to screen and discover new control targets of Pomacea and develop new bio-pesticide to control Pomacea, which is helpful to adjust the current control strategy of Pomacea.

TPR (Tetratricopeptite repeat) genes are a family of genes containing TPR conserved motifs, and this gene is involved in many biological processes such as cell cycle regulation, gene expression, protein transport, mitosis, protein folding, steroid receptor function, RNA splicing, transcription inhibition, protein degradation and stress stress. The TPR gene in Arabidopsis thaliana is mainly involved in the process of high temperature response and growth and development, and the TPR gene in wheat (Triticum aestivum L) plays an important role in response to low temperature and high salt stress. However, there is no report on the study of TPR gene in Pomacea.

TPR1 gene is an important member of TPR gene family, and it is the aptamer protein required by G protein coupling receptor to activate Ras-dependent signaling pathway. Although much research has been done on TPR1 gene and a large number of TPR1 cDNA sequences have been cloned, Pomacea is a worldwide invasive alien organism, and the function and role of TPR1 gene in Pomacea have not been reported. The Pomacea TPR1 gene obtained by the disclosure can lay a foundation for the research on the physiological related genes and functions of aquatic mollusks, and provide a theoretical basis and a practical reference for effectively controlling the spreading hazard of Pomacea.

### SUMMARY

In order to overcome the defects of the prior art, the present disclosure provides a TPR1 gene related to low temperature tolerance of Pomacea, coding protein and application of the TPR1 gene. In the present disclosure, according to the characteristics that the protein coded by the TPR1 gene is relatively conservative but the nucleic acid sequence has lower homology with other organisms, RNA interference is performed on the target gene, the inhibition on the golden apple snails at the nucleic acid level is realized, and the survival rate of the golden apple snails is remarkably reduced.

The specific technical scheme is as follows:

A TPR1 gene related to a low-temperature tolerance of Pomacea, coding protein and application of the gene, wherein the gene has a nucleotide sequence shown in SEQ ID NO: 1 in a sequence table, the gene plays an important role in maintaining the normal low-temperature tolerance of Pomacea, and an inhibition of a function of the gene will result in a decrease of a survival rate of Pomacea.

A TPR1 gene related to a low-temperature tolerance of Pomacea, coding protein and application of the gene, wherein the protein has an amino acid sequence shown in SEQ ID NO:2 in a sequence table, and an inhibition of a function of the gene will result in a decrease of a survival rate of Pomacea.

The application of the TPR1 gene related to a low-temperature tolerance of Pomacea, wherein the application is used for pesticide development and biological control of Pomacea.

The application of a RNA interference technology for the TPR1 gene of Pomacea in the control of Pomacea, wherein the RNA interference technology results in a decrease in the survival rate of Pomacea.

The application of the TPR1 gene related to a low-temperature tolerance of Pomacea, wherein the application is used for pesticide development and biological control of Pomacea.

The advantageous effects of the present disclosure are: the disclosure is rapid, effective and reproducible, and is an important complement to the TPR gene family. By interfering with the expression of the TPR1 gene of the present disclosure and inhibiting the function of the TPR1 gene, the low temperature tolerance of Pomacea can be significantly reduced, the survival rate is reduced, and the prevention and control efficiency is improved, thereby achieving the aim of controlling the further northward invasion of Pomacea. The most important point is that by interfering with the expression of the TPR1 gene of the present disclosure, the hatching rate of the eggs of Pomacea can be significantly reduced, and the incubation period of the eggs can be prolonged. The disclosure is of great significance to the study and determination of the biology, ecology, novel insecticides of Pomacea, and the study of the resistance mechanism of Pomacea to low temperature tolerance.

### BRIEF DESCRIPTION OF THE DRAWINGS

FIG. 1 is an expression of TPR1 in different tissues under different temperature stress;

FIG. 2 Effects of RNA interference on TPR1 gene expression;

FIG. 3 Effects of RNA interference of TPR1 gene on survival rate of Pomacea.

### DETAILED DESCRIPTION OF THE EMBODIMENTS

Hereinafter, that present disclosure will be further described in detail with reference to specific embodiments. In the following embodiments, the materials, methods or other technical features that are not mentioned are consistent with those recorded in the disclosure.

### Embodiment 1

1 Materials and Methods

1.1 Test Pomacea

Before the experiment, all individuals of Pomacea were cultured in a constant temperature laboratory (40 cm×25 cm×28) at a temperature of 26±1° C. and a light of 16 h: 8 h, and the feed is fresh cabbage leaf.

1.2 Main Reagents

Akara MiniBest Universal RNA Extraction Kit TakaRa, TakaRa MiniBEST Agarosc Gel DNA Extraction Kit, Taq enzyme, SYBR Prefix Ex Taq, RACE Kit User Manual were purchased from Clontech Company. USA, and sequencing and primer synthesis were performed by Shanghai Suni Bio-Express Co Ltd.

Cloning of TPR1 Gene from Pomacea

1) taking 10 mg-50 mg of mantle tissue of Pomacea, washing with DEPC water for 3-5 times, and storing in liquid nitrogen;

2) Total RNA extraction: under the condition of liquid nitrogen, grounding the mantle tissue of Pomacea into powder, and then extracting the total RNA of mantle tissue by Takara Minibest Universal RNA Extraction Kit, with specific steps referring to its instructions. Detecting the integrity and purity of RNA by agarose gel electrophoresis and Nanodrop 2000(Thermo).

3) Using 1 μg of total RNA as template, synthesizing the cDNA of Pomacea mantle tissue by using PrimeScript RT reagent reverse transcription and storing at −20° C. for future use.

4) Design merger primers and other primers

Searching the amino acid sequence of TPR1 gene in NCBI database, selecting the amino acid sequence of TPR1 of different species, and then using CODEHOP program to design merger primers.

The main steps of using CODEHOP to design primers are as follows: Saving the amino acid sequences of different species queried above in FASTA format, and submiting the results to Blockmaker (http://blocks.fhcrc.org/blocks/make_blocks.html), searching the conserved region, and then submitting the conserved region to the server for primer design, and the main parameter of the design is Degeneracy 128; the annealing temperature 60° C., the genetic code is standard, and the primer with less degeneracy and suitable Tm value and target fragment length is selected to be sent to the company for synthesis. The designed primer sequences are as follows:

Upstream primer UP1:
5′-TCTCATgaytayctyyt-3′.
Downstream primer UP2:
5′-AGGTCGATACGAGGaacatnaartc-3′.

Note: n is A, T, C or G: Y is C or T; R is A or G.

Designing 3′-RACE and 5′-RACE primers at the same time, and the sequence is as follows:

TPR1-3F:
5′-AATTCGTACAGGCCAAGGCT-3′
TPR1-3R:
5′-TTATCGCTGTCATCGGCTCC-3′
TPR1-5F:
5′-TGCATGCGACTGACTGAAGA-3′
TPR1-5R
5′-CCCATTCGTAGGAGGGTTCTG-3′

5) Cloning of TPR1 Gene from Pomacea

Using the designed merger primers to amplify UP1 and UP2 by PCR, and the intermediate fragment of TPR1 of 357 bp was obtained.

The PCR reaction system was 25 μL: 10xrtaq buffer 2.5 μL, dNTPs (10 nmol/L each) 0.5 μL, MgCl₂ (25 mM) 1.5 μL, cDNA 1 μL, Taq enzyme 0.5 μL, supplemented with ddH₂O water to 25 μL. The PCR procedure was 95° C. for 30 s, 56° C. for 45 s, 72° C. for 1 min, 35 cycles, 72° C. extended for 10° C.

Using TPR1-3F and TPR1-3R primer pairs, the 3′end fragment of 1100 bp was amplified.

Using TPR1-5F and TPR1-5R primer pairs, the 5′end fragment of 625 bp was amplified.

In order to obtain the full-length TPR1 gene of Pomacea (873 bp), the amplified fragment was spliced by DNAman software. SEQ ID NO:1 and SEQ ID NO:2 respectively provide the nucleotide sequence and the amino acid sequence of the full-length ORF gene.

### Embodiment 2

Analysis of TPR1 Expression in Different Tissues Under Different Temperature Stress

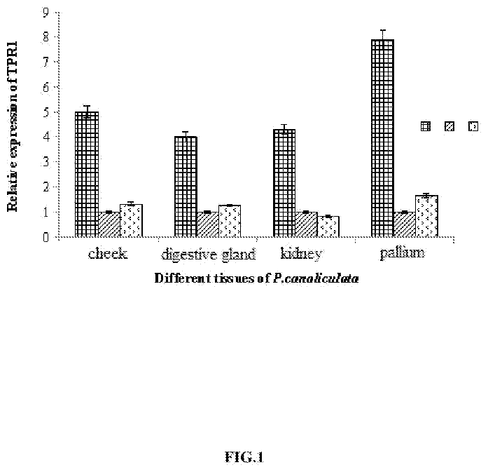

Total RNA was extracted from different tissues and the expression of TPR1 gene was detected by qPCR under different temperature stress. The reaction system was 25 μL. The reaction procedure was as follows: 95° C. pre-denatured for 1 min, then 95° C. for 30 s, 58° C. for 45 s, 72° C. for 1 min, 35 cycles; all data were analyzed by Excel 2010. The relative expression level of the TPR1 gene was analyzed according to the Ct method (2-.DELTA.Ct method). All data are labeled as mean ±SE. the results are shown in FIG. 1.

As can be seen from FIG. 1, under the heat shock conditions (36° C.), there was little change in the individual tissues of TPR1 compared to the control (26° C.). The expression level of TPR1 was significantly increased in all the tested tissues (P<0.05) under the cold shock condition (6° C.), and the expression level was highest in the mantle tissues. Therefore, under the condition of short-term cold stress, TPR1 gene is the cold shock metabolite of Pomacea, which plays an important role in improving the low temperature tolerance of Pomacea.

### Embodiment 3: Effect of TPR1-dsRNA from Pomacea on mRNA Expression of Target Gene

(1) TPR1-dsRNA of Pomacea obtained by the disclosure was transfected into Pomacea by injection method, and was continuously fed at 0° C. for 5 days.

(2) Total RNA was extracted from the mantle tissues of Pomacea at 0, 6, 12, 24, 72 and 120 hours after transfection, and the expression level of TPR1 gene mRNA was detected by real-time PCR.

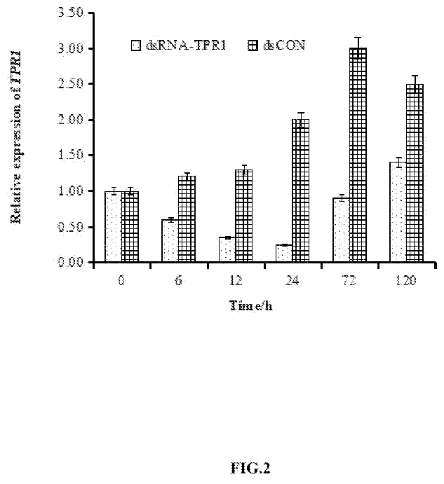

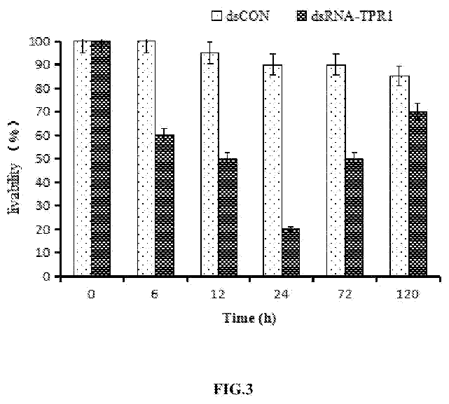

Results as shown in FIG. 2, the expression level of TPR1 in the experimental group decreased significantly 6 hours after transfection, reached the lowest point 24 hours after transfection, and the interference effect decreased with the increase of transfection time, and the expression level of TPR1 in the experimental group began to increase. However, the expression of TPR1 in the experimental group was significantly lower than that in the control group (P<0.05). The construction of the small interfering RNA expression vector of Pomacea TPR1 constructed by the present disclosure is successful. As can be seen from FIG. 3, with the increase of transfection time, the expression level of TPR1 gradually decreased, and the survival rate of Pomacea lost the protection of TPR1. The survival rate of Pomacea increased slowly with the failure of dsRNA-TPR1 interference.

### Embodiment 4: Effect of Pomacea TPR1-dsRNA on the Hatchability of Pomacea Eggs

TPR1-dsRNA of Pomacea obtained by the disclosure was transfected into mature female and male Pomacea snails by injection method, the female and male snails were continuously fed at 26° C. for 6 days, after the female snails lay eggs, the egg pieces were transferred to an incubator, and the hatching of the egg pieces was observed and recorded. The illumination of the incubator is L: D=13:11, and the incubation temperature is 26° C. The hatching rate and incubation period of each egg block were observed every day. The results showed that the average incubation period was 18.8 days and the average hatching rate was 78.3%, while the average incubation period was 23.4 days and the average hatching rate was 25.3%. It shows that interfering with the expression of TPR1 gene of Pomacea can significantly reduce the hatching rate of Pomacea eggs and prolong the hatching period of eggs, which provides theoretical basis and practical reference for controlling the further invasion of Pomacea and developing environmentally friendly bio-source pesticides.

What is claimed is:

1. A TPR1 gene related to a low-temperature tolerance of Pomacea, coding protein and application of the gene, wherein the gene has a nucleotide sequence shown in SEQ ID NO:1 in a sequence table, the gene plays an important role in maintaining the normal low-temperature tolerance of Pomacea, and an inhibition of a function of the gene will result in a decrease of a survival rate of Pomacea.

2. A TPR1 gene related to a low-temperature tolerance of Pomacea, coding protein and application of the gene, wherein the protein has an amino acid sequence shown in SEQ ID NO:2 in a sequence table, and an inhibition of a function of the gene will result in a decrease of a survival rate of Pomacea.

3. The application of claim 1, wherein the application is used for pesticide development and biological control of Pomacea.

4. The application of a RNA interference technology for the TPR1 gene of Pomacea in the control of Pomacea of claim 1, wherein the RNA interference technology results in a decrease in the survival rate of Pomacea.

5. The application of claim 2, wherein the application is used for pesticide development and biological control of Pomacea.
